(19) United States
(12) Patent Application Publication
(10) Pub. No.: US 2009/0296949 A1
(43) Pub. Date: Dec. 3, 2009

(54) ACOUSTIC CHARACTERISTIC CORRECTION APPARATUS, ACOUSTIC CHARACTERISTIC MEASUREMENT APPARATUS, AND ACOUSTIC CHARACTERISTIC MEASUREMENT METHOD

(76) Inventors: Shigeyasu Iwata, Hamura-shi (JP); Yutaka Oki, Kawasaki-shi (JP); Takashi Fukuda, Ome-shi (JP); Toshifumi Yamamoto, Sagamihara-shi (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/473,758

(22) Filed: May 28, 2009

(30) Foreign Application Priority Data
May 29, 2008 (JP) 2008-141479

(51) Int. Cl. A61F 11/06

(52) U.S. Cl. 381/71.6

(57) ABSTRACT

According to one embodiment, an acoustic characteristic measurement apparatus comprises a generation module configured to generate a measuring pulse for measuring an acoustic characteristic of a subject, an acquisition module configured acquire an acoustic characteristic of the subject based on a response signal from the subject, and a transducer module configured to transduce the measurement pulse into an acoustic signal to be transmitted to the subject and transduce an acoustic response signal from the subject into an electrical response signal to be supplied to the acquisition module.

### CROSS-REFERENCE TO RELATED APPLICATIONS

This application is based upon and claims the benefit of priority from Japanese Patent Application No. 2008-141479, filed May 29, 2008, the entire contents of which are incorporated herein by reference.

### BACKGROUND

1. Field

One embodiment of the invention relates to an acoustic characteristic correction apparatus, an acoustic characteristic measurement apparatus, and an acoustic characteristic measurement method which measure and correct resonance characteristic of subjects to be measured, for example, an outer-ear canal of a listener who listens to a sound source signal.

2. Description of the Related Art

When listening to music through earphones or headphones (hereinafter, both are referred to as earphones), a resonance phenomenon occurs between the eardrum and an earphone because the outer-ear canal is filled with the earphone, a sound of a resonance frequency is emphasized, and then, an unnatural sound may be generated. Therefore, it is preferable to measure resonance characteristic in the outer-ear canal and correct external canal resonance characteristic at a time of listening to a sound source signal.

There is an individual difference in a shape and an acoustic transmission characteristic of the outer-ear canal, and in physicality and in an acoustic transmission characteristic of the eardrum. Further, the resonance in the outer-ear canal depends on an individual due to a kind of the earphone and a wearing state thereof. Therefore, it is necessary to individually correct outer-ear canal resonance characteristic in order to accurately correct the resonance in the outer-ear canal (refer to Jpn. Pat, Appln. KOKAI Publication No. 2001-285998, paragraphs [0012], [0013], and [0014]).

An out-of-head sound image localization device which perceives a position (sound image) to be perceived as a sound source outside a head by using acoustic equipment, which contacts with both ears, such as binaural headphones and binaural earphones, is disclosed in the above patent document. The sound source signal is supplied to the earphone through a digital filter. While a transmission function (out-of-head sound image localization transmission function [SLTF]) of the digital filter may be obtained on a calculator, each of outer-ear canal transmission functions (ECTFs) and each of head-related acoustic transmission functions (HRTFs) (SLTF is nearly equal to HRTF and/or ECTF) are each different depending on sizes of the outer-ear canals, sizes of ears and sizes of faces of the listeners. Namely, only a transmission function coinciding with a shape of a face of an individual enables accurately localizing the sound image outside the head, enables determining front and behind of the sound image, and enables localizing the sound image outside the head.

Therefore, since the transmission functions cannot be generally utilized for unspecified listeners if the sound images are not localized outside the heads depending on differences among individuals of the transmission functions, a resolution method as hardware for selecting a several kinds of transmission functions which have been measured in advance, or a system for measuring a transmission function for each individual person is needed. However, there are problems that the foregoing resolution method as hardware poses an increase in hardware volume, and that the system for measuring the transmission function for each individual person makes a cost of such a measuring system very expensive.

Thus, the out-of-head sound image localization device disclosed in the foregoing patent document provides an adaptive filter in order to obtain a reverse function of the outer-ear canal transmission function (ECTF) that is a transmission function from the earphone to a microphone output, and supplies the sound source signal to the adaptive filter through the digital filter. Meanwhile, a band pass filter composed so that a product between the outer-ear canal transmission function and the reverse transmission function of the adaptive filter becomes filter characteristic is connected to an output from the digital filter. An output from the adaptive filter is supplied to the earphone. A subtracter performs a subtraction between the output from the microphone which picks up the output from the earphone and the output from the band pass filter, a convergence calculation circuit receives the subtraction result and the calculation circuit converges the reverse transmission function of the adaptive filter based on the subtraction result.

The device described in the aforementioned patent document needs a microphone in addition to an earphone in order to measure the acoustic characteristic of the outer-ear canal of the listener then the microphone is attached to the earphone. Thereby, the earphone becomes large and complex.

In this way, since it is necessary to attach the microphone to the earphone so as to measure the outer-ear canal resonance characteristic in the conventional manner, the earphone itself becomes large and complex, the acoustic characteristic of the outer-ear canal of the listener cannot be accurately measured at ease, it is impossible to obtain and precisely correct the acoustic characteristic.

### BRIEF DESCRIPTION OF THE SEVERAL VIEWS OF THE DRAWINGS

A general architecture that implements the various feature of the invention will now be described with reference to the drawings. The drawings and the associated descriptions are provided to illustrate embodiments of the invention and not to limit the scope of the invention.

FIG. 1 is an exemplary view depicting a concept of an outer-ear canal characteristic correction according to a first embodiment of the invention;

FIG. 2 is an exemplary block diagram depicting a configuration example of an outer-ear canal characteristic correction apparatus 40 according to the first embodiment of the invention;

FIG. 3 is an exemplary flowchart depicting an example of operations of the correction apparatus 40 shown in FIG. 2;

FIGS. 4A, 4B and 4C are exemplary views depicting examples of switching operations of a selection module 42 of the correction apparatus 40 shown in FIG. 2;

FIGS. 5A, 5B and 5C are exemplary views depicting modification examples of switching operations of the selection module 42 of the correction apparatus 40 shown in FIG. 2;

FIG. 6 is an exemplary view depicting a quasi-outer-ear canal for use in an experiment showing that frequency characteristic of an outer-ear canal which have been picked up near a eardrum and an earphone coincide with each other;

FIG. 7 is an exemplary view depicting frequency characteristic depicting an experiment result;

FIG. 8 is an exemplary view depicting an implementation example of the first embodiment;

FIG. 9 is an exemplary block diagram depicting a configuration example of an outer-ear canal characteristic correction apparatus according to a second embodiment of the invention;

FIG. 10 is an exemplary block diagram depicting a configuration example of an outer-ear canal characteristic correction apparatus according to a third embodiment of the invention;

FIG. 11 is an exemplary flowchart depicting an operation example of the outer-ear canal characteristic correction apparatus according to the third embodiment shown in FIG. 10;

FIG. 12 is an exemplary block diagram depicting a configuration example of an outer-ear canal characteristic correction apparatus according to a fourth embodiment of the invention;

FIG. 13 is an exemplary block diagram depicting a configuration example of an outer-ear canal characteristic correction apparatus according to a fifth embodiment of the invention;

FIG. 14 is an exemplary flowchart depicting an operation example of the outer-ear canal characteristic correction apparatus regarding the fifth embodiment of the invention;

FIG. 15 is an exemplary block diagram depicting a configuration example of an outer-ear canal characteristic correction apparatus according to a sixth embodiment of the invention;

FIG. 16 is an exemplary block diagram depicting a configuration example of an outer-ear canal characteristic correction apparatus according to a seventh embodiment of the invention;

FIG. 17 is an exemplary flowchart depicting an operation example of the outer-ear canal characteristic correction apparatus according to the seventh embodiment of the invention; and

FIG. 18 is an exemplary block diagram depicting a configuration example of an outer-ear canal characteristic correction apparatus according to an eighth embodiment of the invention.

### DETAILED DESCRIPTION

Various embodiments according to the invention will be described hereinafter with reference to the accompanying drawings. In general, according to one embodiment of the invention, an acoustic characteristic measurement apparatus comprises a generation module configured to generate a measuring pulse for measuring an acoustic characteristic of a subject; an acquisition module configured acquire an acoustic characteristic of the subject based on a response signal from the subject; and a transducer module configured to transduce the measurement pulse into an acoustic signal to be transmitted to the subject and transduce an acoustic response signal from the subject into an electrical response signal to be supplied to the acquisition module.

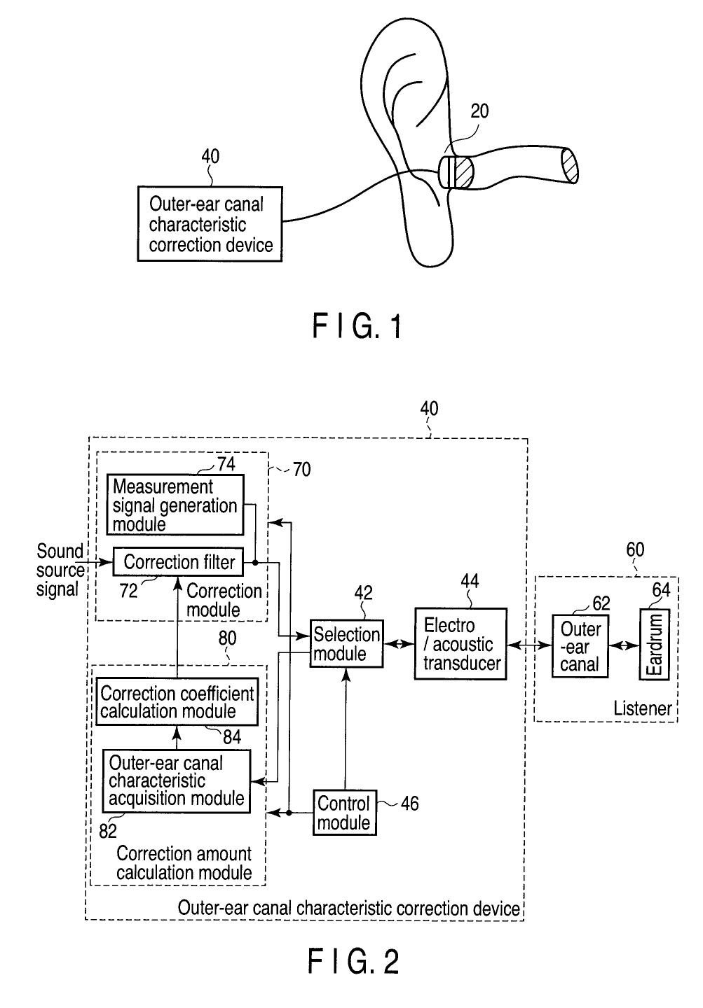

FIG. 1 shows a view depicting a concept of correction of outer-ear canal resonance characteristic as an acoustic transmission characteristic in an embodiment of the invention. An end part of an outer-ear canal of a listener is filled with an earphone 20, an outer-ear canal characteristic correction apparatus 40 is arranged outside the outer-ear canal, and both the earphone 20 and the correction apparatus 40 are electrically connected. Since the right and the left outer-ear canals are different in characteristic, although not shown, each of the correction apparatuses 40 is connected to the right and the left earphones 20, and the right and the left characteristic are corrected, respectively.

FIG. 2 shows a view depicting a configuration example of the correction apparatus 40. The correction apparatus 40 includes a correction module 70, a correction amount calculation module 80, a selection module 42, a control module 46 and an electro/acoustic transducer 44. A listener 60 includes an outer-ear canal 62 and an eardrum 64.

The electro/acoustic transducer 44 is an earphone 20 or a headphone filling the end part of the outer-ear canal as shown in FIG. 1, and transduces an electrical signal output from the selection module 42 into an acoustic signal to be supplied to the outer-ear canal 62. The transducer 44 also transduces the acoustic signal, which is output to the outer-ear canal 62 and reflected from the eardrum 64 to be returned to the transducer 44, into the electrical signal to be supplied to the selection module 42.

Since the outer-ear canal 62 is filled with the earphone (electro/acoustic transducer 44), the acoustic signal output to the outer-ear canal from the transducer 44 is resonant. The correction apparatus 40 detects a resonance frequency to correct (reduce a gain of the resonance frequency) frequency characteristic of an acoustic signal corresponding to a sound source signal to be output to the outer-ear canal 62 from the earphone 20.

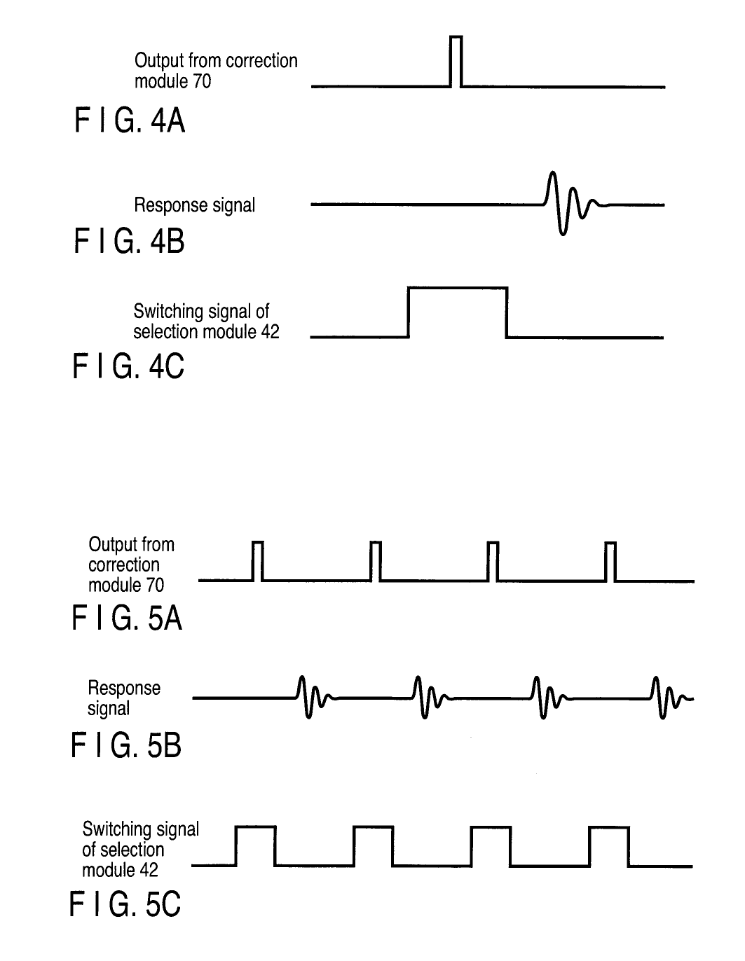

Under the control by the control module 46, the selection module 42 is brought into any one of a first state and a second state. In the first state, the selection module 42 connects the correction module 70 to the transducer 44 to output the electrical signal that is an output from the correction module 70 to the outer-ear canal 62 as the acoustic signal through the transducer 44. In the second state, the selection module 42 connects the correction amount calculation module 80 to the transducer 44 to output the electrical signal that is an output from the transducer 44 to the calculation module 80. FIGS. 4A, 4B and 4C show each selection operation of the selection module 42. The control module 46 supplies a switching signal shown in FIG. 4C to the selection module 42. If the switching signal has a high level, the selection module 42 is brought into the first state, and if the switching signal has a low level, the selection module 42 is brought into the second state. Although not shown, the selection module 42 is brought into the first state at a time of listening to the sound source signal.

The correction module 70 includes a measurement signal generation module 74 which generates a unit pulse or a time stretched pulse (TSP) within a prescribed width that is a reference signal for measurement to measure an acoustic characteristic (resonance frequency) of the outer-ear canal 62; and a correction filter 72 correcting a sound source signal heard by the listener 60.

The correction apparatus 40 is switched into an acoustic characteristic measurement mode and a sound source signal correction (sound source signal listening) mode. In the acoustic characteristic measurement mode, the correction module 70 outputs an output from the generation module 74, and in the sound source signal correction mode, the correction module 70 outputs an output from the correction filter 72. FIGS. 4A, 4B and 4C show each switching operation of the selection module 42 in the sound source signal correction mode. In the acoustic characteristic measurement mode, the unit pulse or TSP that is a reference signal for measurement within the prescribed width is supplied to the transducer 44 through the selection module 42 to be transduced into the acoustic signal, and the acoustic signal is output to the outer-ear canal 62. After this, as shown in FIG. 4C, the selection module 42 is switched on the side of the calculation module 80, the acoustic signal (response signal) reflected by the eardrum 64 is transduced again into the electrical signal through the transducer 44, and the electrical signal (response signal) is input to the calculation module 80.

The calculation module 80 includes an outer-ear canal characteristic acquisition module 82 receiving the response signal to acquire an acoustic characteristic (resonance frequency) of the outer-ear canal 62; and a correction coefficient calculation module 84 calculating a correction coefficient of the correction filter 72 based on the acoustic characteristic acquired from the acquisition module 82.

The control module 46 controls the correction module 70 in response to the acoustic characteristic measurement mode or the sound source correction mode, and also switches to control the selection module 42 in response to the first state or the second state as described above.

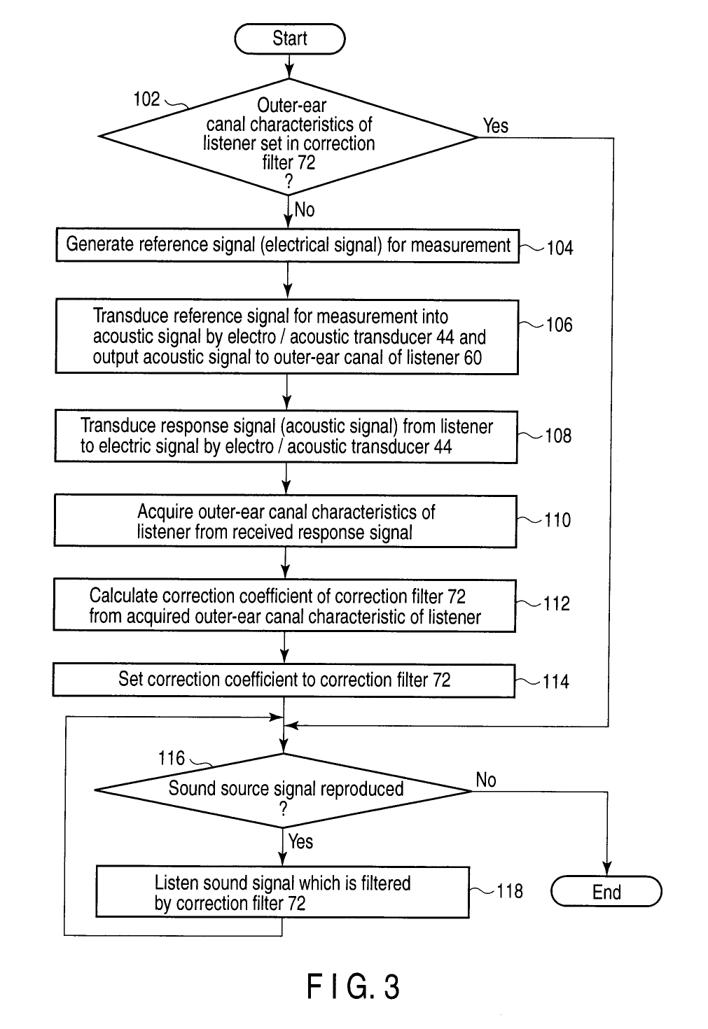

FIG. 3 shows a flowchart depicting operations of the control module 46 of the outer-ear canal characteristic correction apparatus 40 of FIG. 2. In an initial state, an operation mode is brought into the acoustic characteristic measurement mode. It is determined whether or not a correction coefficient corresponding to outer-ear canal characteristic of the listener 60 is set in the correction filter 72 (Block 102). If the correction coefficient is set, it is determined whether or not the operation mode is the sound source signal correction (sound source signal listening) mode (Block 116). If the operation mode is not in the sound source signal correction mode, the control module 40 ends its operation.

If it is determined that the correction coefficient corresponding to the outer-ear canal characteristic of the listener 60 is not set in the correction filter 72 (Block 102), the correction module 70 selects the output from the measurement signal generation module 74 (Block 104). Thereby, the reference signal for measurement is generated from the generation module 74 to be supplied to the selection module 42. At this moment, the selection module 42 has been brought into the first state as shown in FIG. 4C, the output (reference signal for measurement) from the correction module 70 (FIG. 4A) is output into the outer-ear canal 62 as the acoustic signal through the transducer 44 (Block 106). As shown in FIG. 4C, after this, the selection module 42 is switched into the second state, the response signal that is the reflection signal of the acoustic signal to the reference signal for measurement reflected by the eardrum 64 (FIG. 4B) is transduced into an electrical signal through the transducer 44 to be input in the calculation module 80 (Block 108).

The acquisition module 82 acquires the outer-ear canal resonance characteristic in response to the received response signal (Block 110). The calculation module 84 (i) transforms the acquired outer-ear canal resonance characteristic from a time domain into a frequency domain, (ii) detects resonance peaks on a frequency axis, and (iii) calculates a coefficient of the correction filter so as to form dips of frequencies at which peaks are generated for canceling the detected peaks (Block 112). The calculation of the coefficient may be performed by using a parametric equalizer and a graphic equalizer. The coefficient is set to the correction filter 72 (Block 114). After this, a process in Block 116 is performed, it is determined whether or not the operation mode is the sound source signal correction (sound signal listening) mode.

If it is determined that the operation mode is the sound source signal correction (sound source listening) mode (Block 116), the correction module 70 selects the output from the correction filter 72 (Block 118). Thereby, the sound source signal with filtering processing through the correction filter 72 processed thereto is supplied to the selection module 42. At this moment, the selection module 42 has been brought into the first state, and the sound source signal after resonance characteristic correction is output into the outer-ear canal 62 as the acoustic signal through the transducer 44.

In this way, creating a filter which cancels the resonance peaks which have been actually measured by an outer-ear canal of each person and performing filtering processing for the right and the left sound source signals smooth the resonance peaks shown in FIG. 7 even if resonance is caused in the outer-ear canal, the resonance peaks shown in FIG. 7 are smoothed, and the listener is prevented from listening to an unnatural sound. Further, since the microphone has not been attached to the earphone, the earphone is not made larger in size, and is not made complex in structure.

While the aforementioned embodiment has been described that the reference signal for measurement is output one time, and the outer-ear canal characteristic are acquired based on the response signal, since sensitivity of the earphone is lower than that of the microphone, the level of the response signal is lowered and masked by noise, and the acoustic characteristic may not be measured. In such a case, the control module 46 switches itself at a plurality of times to output the reference signals for measurement at a plurality of times, as shown in FIGS. 5A to 5C, repeats a plurality of times of acquisition of the acoustic characteristic to average a plurality of response signals, and then, accurate an acoustic characteristic from which influence of the noise is eliminated may be measured.

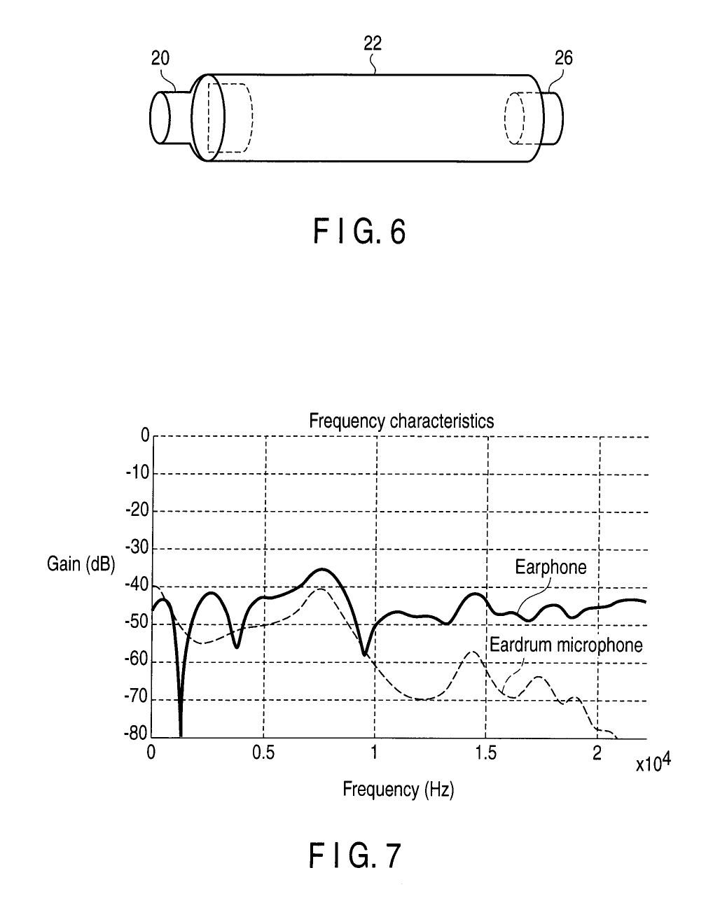

The fact that the frequency characteristic which have been picked up near the eardrum and the frequency characteristic which have been picked up at a position (near the earphone) not at the eardrum coincide with each other will be described with reference to FIGS. 6 and 7. FIG. 6 shows a view illustrating an experiment outline using a quasi-outer-ear canal 22. The quasi-outer-ear canal 22 is a cylindrical tube which imitates a human outer-ear canal. An experiment is performed by attaching an eardrum microphone 26 and an earphone 20 at opposite ends of the quasi-outer-ear canal 22. The earphone 20 outputs a unit pulse or a TSP within a prescribed width, the earphone 20 and the microphone 26 pick up the pulses, and the experiment compares the frequency spectra with each other.

FIG. 7 shows a view illustrating the frequency characteristic of the microphone 26 and the earphone 20 which have been obtained in the experiment. As shown in FIG. 7, while the characteristic obtained through the earphone 20 produces dips at which nodes of the standing wave are positioned, frequencies (near 7.5 KHz, and 15 KHz) at which resonance peaks are generated are nearly coincide with characteristic obtained through the microphone 26. Thereby, since the frequency characteristic measured with the earphone 20 varies in accordance with an attachment position of the earphone 20, a correct inverse filter cannot be made even if an inverse filter of the obtained frequency characteristic has been made and then it is hard to correctly cancel resonance phenomena. However, since the resonance frequencies are correct, correcting by using solely these resonance frequencies enables correctly canceling the resonance phenomena.

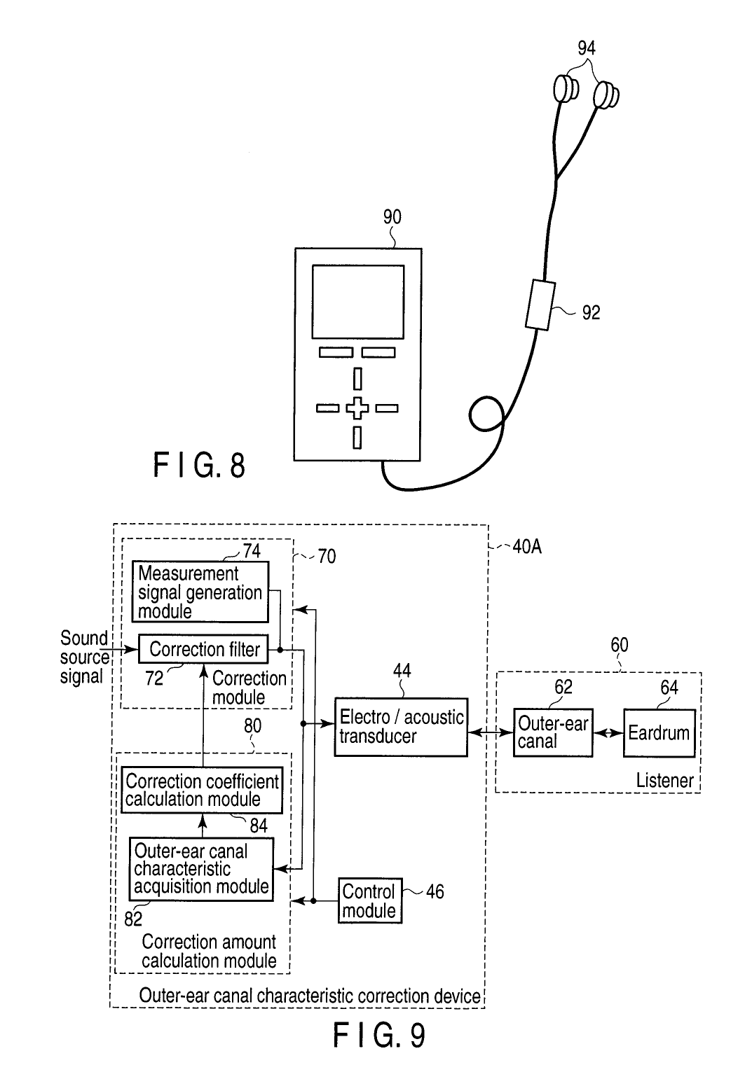

FIG. 8 shows an implementation example of the correction apparatus 40 of FIG. 2. For installing the correction apparatus 40 in an audio player 90, the position is not limited to a main unit of the audio player 90; the correction apparatus 40 may be installed in a remote controller 92 or an earphone 94. Instead of installing the whole of the correction apparatus 40 in the audio player 90, solely the correction filter 72 may be installed therein. That is, from the generation of the reference signal for the measurement up to the measurement of the outer-ear canal characteristic and the calculation of the correction coefficient are performed through a separated PC, etc., and the audio player 90 uses the correction filter 72 with the obtained correction coefficient applied thereto, and may only correct the sound source signal read from a flash memory, a hard disk, etc., (not shown). Or, for installing the correction apparatus 40 in the audio player 90, it is also able to correct the sound source signal when it is downloaded and to store the corrected sound signal in a memory, etc.

While the aforementioned explanation has been described in the case where the microphones are attached to the right and the left earphones and obtains the characteristic of the right and the left ears to create correction filters for each of the characteristic, the correction apparatus 40 may be configured to obtain characteristic of solely one ear, and use a correction filter created by using the characteristic to filter the sound source signals for both the ears.

Since the resonance characteristic may be varied in response to the positions of the earphones, the acoustic characteristic measurement and the acoustic characteristic correction processing by means of the correction apparatus 40 may be performed, for example, at every time when the audio player 90 is started, may be performed at a time when a user arbitrarily operates the player 90, or may be performed at a time when the player 90 is started after the period specified by the user has passed.

As described above, according to the embodiment, the outer-ear canal characteristic correction apparatus 40 supplies the reference signal for measurement to the electro/acoustic transducer 44 through the selection module 42 to output the acoustic signal, transduces the sound signal reflected from the eardrum into the electrical signal through the transducer 44, and supplies the electrical signal to the correction amount calculation module 80. Thereby, the correction apparatus 40 obtains the resonance frequencies in the outer-ear canal, calculates the correction coefficient of the correction filter 72 so as to cancel the resonance frequencies and then may accurately cancel the resonance phenomena of each person's external ear acoustic characteristic with a simple configuration without setting the microphone near the earphone. Further, individually obtaining the resonance characteristic generated at the earphones and the eardrums, creating the correction filters matching with the characteristic enables canceling the outer-ear canal resonance characteristic differing from one another depending on the outer-ear canal characteristic and insertion states for each individual. Obtaining the characteristic of both the right and left ears and creating the correction filters for each of the characteristic enables canceling the outer-ear canal resonance characteristic differing from the right and the left ears.

The following will describe other embodiments of the invention. In the description of other embodiments, the same components as those of the first embodiment are designated by the identical symbols and the detailed descriptions therefor will be omitted.

FIG. 9 shows a block diagram of an outer-ear canal characteristic correction apparatus 40A of a second embodiment. The correction apparatus 40A of the second embodiment is a correction apparatus which is configured by removing the selection module 42 from the correction apparatus 40 of the first embodiment. Thereby, the output from the correction module 70 is supplied to the calculation module 80 as well as to the electro/acoustic transducer 44. However, since the control module 46 controlling operation timing recognizes whether the switching signal shown in FIG. 4C has a high level (a period in which the reference signal for measurement is output from the correction module 70) or has a low level (a reception period of the response signal from the outer-ear canal 62), the calculation module 80 may separate based on the signal from the control module 46, in chronological order, the reference signal for measurement output from the correction module 70 and the response signal that is the reflection signal from the eardrum 64, and obtain the outer-ear canal characteristic, correctly based on the response signal output from the transducer 44 and not based on the reference signal output from the correction module 70 of the calculation module 80.

In this way, according to the second embodiment, the correction apparatus 40A can input the response signal to the reference signal in the outer-ear canal characteristic acquisition module 82 at correct timing without having to be provided with the selection module 42, and correctly acquire the outer-ear canal characteristic.

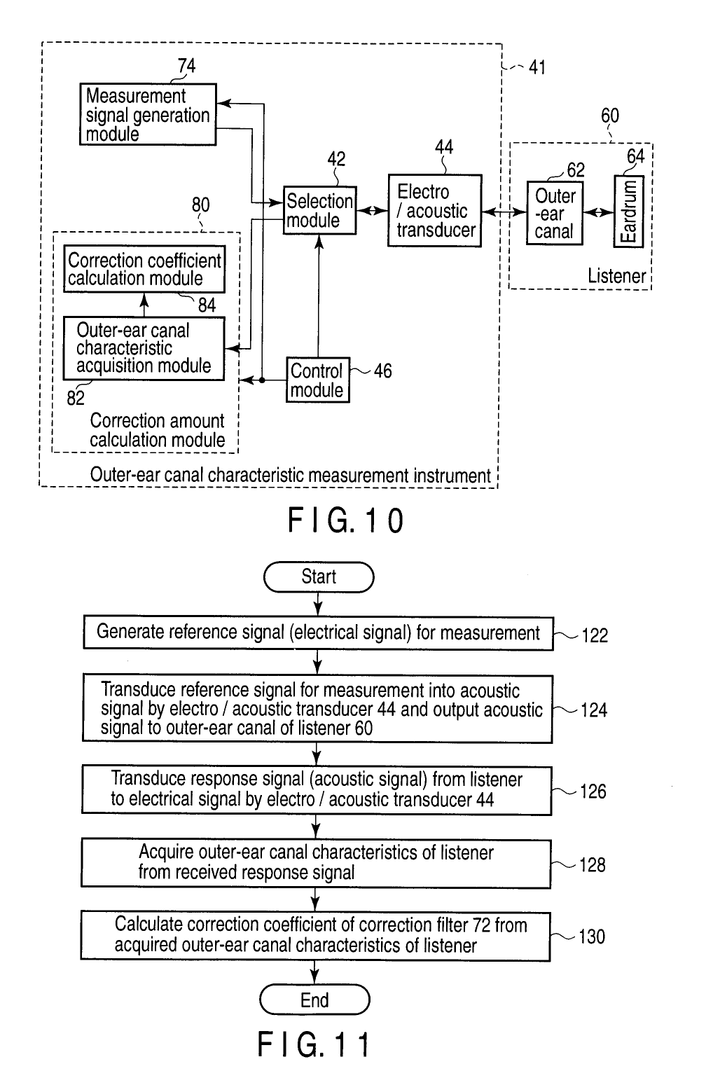

FIG. 10 is a block diagram of an outer-ear canal characteristic measurement apparatus 41 of a third embodiment. The apparatus 41 of the third embodiment is an apparatus which is configured by removing the correction filter 72 from the correction apparatus 40 of the first embodiment.

FIG. 11 shows a flowchart depicting operations of the control module 46 of the measurement apparatus 41. The measurement signal generation module 74 generates the reference signal for measurement to supply it to the selection module 42 (Block 122). At this moment, the selection module 42 has been brought into the first state as shown in FIG. 4C, the output from the generation module 74 (reference signal for measurement) (FIG. 4A) is output into the outer-ear canal 62 as the acoustic signal through the transducer 44 (Block 124). The selection module 42, as shown in FIG. 4C, is switched into the second state after this, the response signal that is the reflex signal of the acoustic signal to the reference signal reflected from the eardrum 64 (FIG. 4B) is transduced into the electrical signal through the transducer 44 to be input in the calculation module 80 (Block 126).

The acquisition module 82 acquires the outer-ear canal resonance characteristic in response to the received response signal (Block 128). The correction coefficient calculation module 84 (i) transforms the acquired resonance characteristic from the time domain into the frequency domain, (ii) detects the resonance peaks on the frequency axis, and (iii) calculates the coefficient of the correction filter so as to form dips of frequencies at which peaks are generated in order to cancel the detected peaks (Block 130). The calculation of the correction coefficient may be performed by using a parametric equalizer or a graphic equalizer.

The calculated correction coefficient is set in the correction filter of the separately disposed correction apparatus.

Thereby, a measurement apparatus which measures resonance characteristic and calculates the correction coefficient of the correction filter, and a correction apparatus to which the calculated correction coefficient is prepared to filter the sound source signal may be structured as separate units.

The measurement apparatus 41 may not include the calculation module 84. In this case, the flowchart of FIG. 11 performs up to the acquisition of the resonance characteristic in Block 128, and the correction coefficient calculation module included in another apparatus calculates the correction coefficient which should be performed in Block 130.

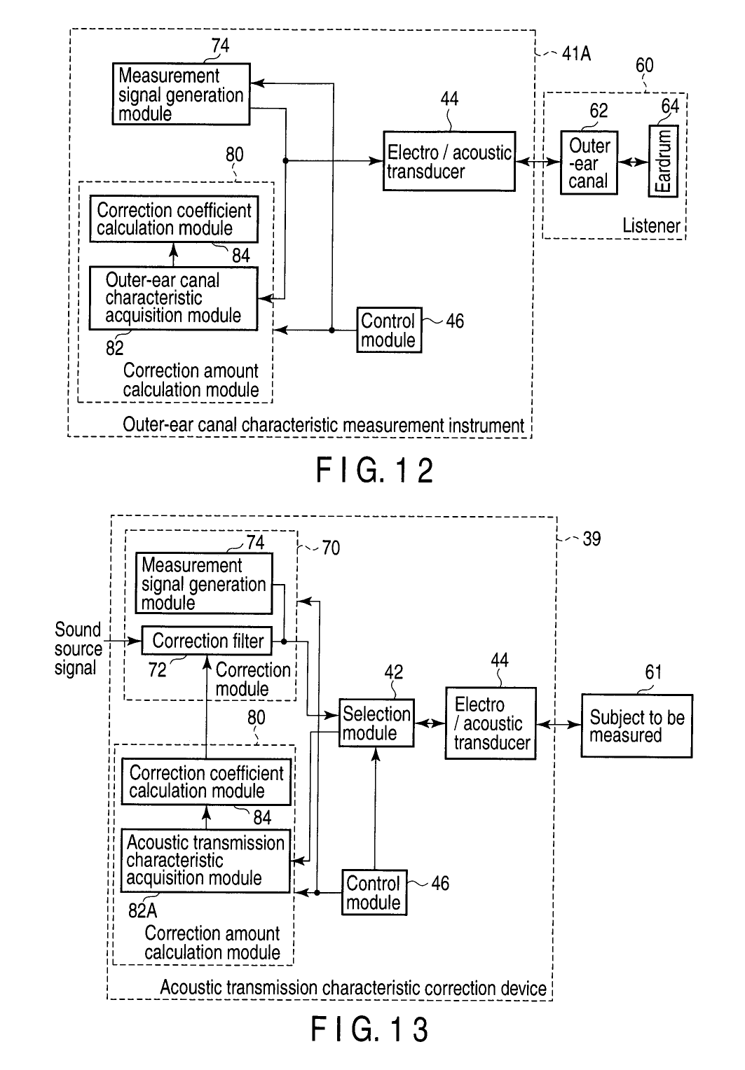

FIG. 12 shows a block diagram of an outer-ear canal characteristic measurement apparatus 41A of a fourth embodiment. The measurement apparatus 41A of the fourth embodiment is configured by removing the selection module 42 from the measurement apparatus 41 of the third embodiment. In the same way as that of the second embodiment, also in the fourth embodiment, the correction amount calculation module 80 separates based on the signal from the control module 46, in chronological order, the reference signal for measurement output from the measurement signal generation module 74 and the response signal that is the reflection signal from the eardrum 64 to the reference signal. The calculation module 80 may correctly receive the response signal from the transducer 44 to correctly acquire the outer-ear canal resonance characteristic.

In the same way as that of the third embodiment, the measurement apparatus 41A may not include the calculation module 84.

While the foregoing embodiments have been described in the cases where the subjects to be measured are the outer-ear canal resonance characteristic of the listeners, embodiments which are not limited to the foregoing embodiments will be described hereinafter.

FIG. 13 shows a view depicting a configuration example of an acoustic transmission characteristic correction apparatus 39 of a fifth embodiment concerning a subject 61 to be measured instead of the listener 60. Although the acoustic transmission function correction apparatus 39 is nearly equal to the correction apparatus 40 of the first embodiment, instead of the outer-ear canal characteristic acquisition module 82, an acoustic transmission characteristic acquisition module 82A is disposed in the correction apparatus 39.

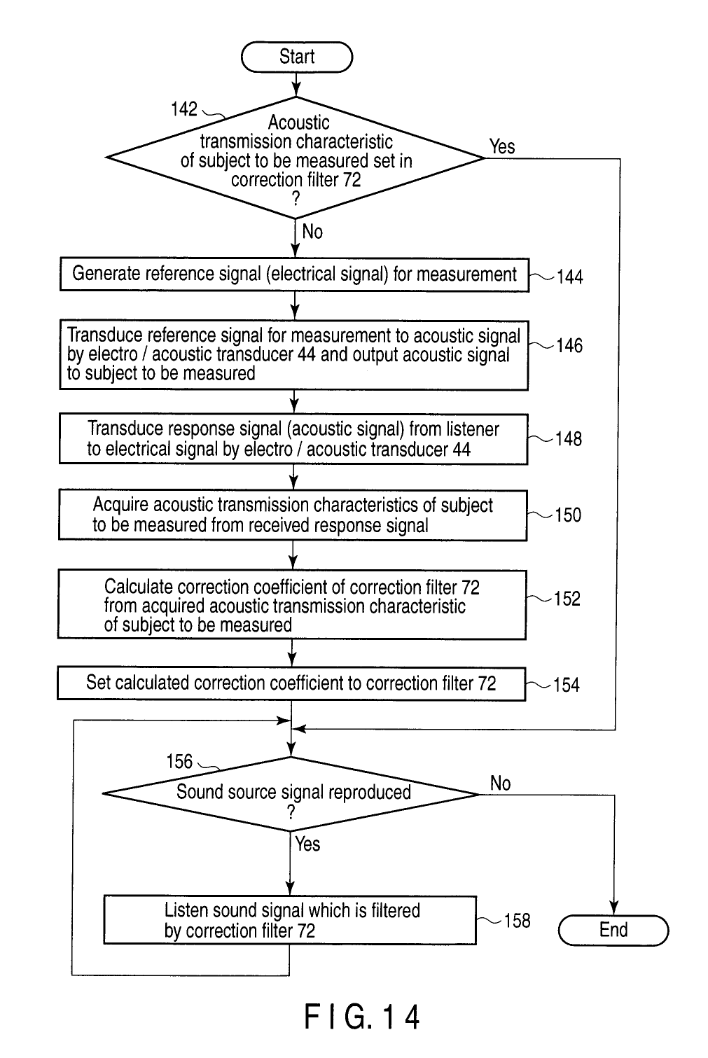

FIG. 14 shows a flowchart illustrating operations of the control module 46 of the correction apparatus 39. In an initial state, an operation mode has been brought into an acoustic characteristic measurement mode. It is determined whether or not a correction coefficient corresponding to an acoustic transmission characteristic of the subject 61 to be measured has been set in the correction filter 72 (Block 142). If the coefficient has been set therein, it is determined whether or not the operation mode is a sound source signal correction mode (Block 156). If the operation mode is not in the sound source signal correction mode, the flowchart comes to an end.

If the correction coefficient corresponding to the acoustic transmission characteristic of the subject 61 to be measured has not been set in the correction filter 72 (Block 142), the correction module 70 selects the output from the measurement signal generation module 74 (Block 144). Thereby, the generation module 74 generates the reference signal for measurement to be supplied to the selection module 42. At this moment, the selection module 42 has been brought into the first state as shown in FIG. 4C, the output (reference signal for measurement) from the correction module 70 (FIG. 4A) is output into the subject 61 to be measured as the acoustic signal through the transducer 44 (Block 146). As shown in FIG. 4C, the selection module 42 is switched into the second state, the response signal that is the reflection signal of the acoustic signal to the reference signal reflected from any portion of the subject 61 to be measured (FIG. 4B) is transduced into the electrical signal through the transducer 44 to be input in the correction amount calculation module 80 (Block 148).

The acquisition module 82A acquires the resonance characteristic in response to the received response signal (Block 150). The calculation module 84 (i) transforms the acquired acoustic transmission characteristic from the time domain into the frequency domain, (2) detects the resonance peaks on the frequency axis, and (3) calculates the coefficient of the correction filter so as to form the dips of the frequencies at which peaks are generated in order to cancel the peaks (Block 152). The calculation of the coefficient may be performed by using the parametric equalizer and the graphic equalizer. The correction coefficient is set in the correction filter 72 (Block 154). After this, the process of Block 156 is performed, and it is determined whether or not the operation mode is the sound source signal correction mode.

If the operation mode is the sound source signal correction mode (Yes in Block 156), the correction module 70 selects the output from the correction filter 72 (Block 158). Thereby, the sound source signal with the filtering processing through the correction filter 72 performed thereto is supplied to the selection module 42. At this moment, the selection module 42 has been brought into the first state and the sound source signal which has been corrected its acoustic transmission characteristic is output to the subject 61 to be measured as the acoustic signal through the transducer 44.

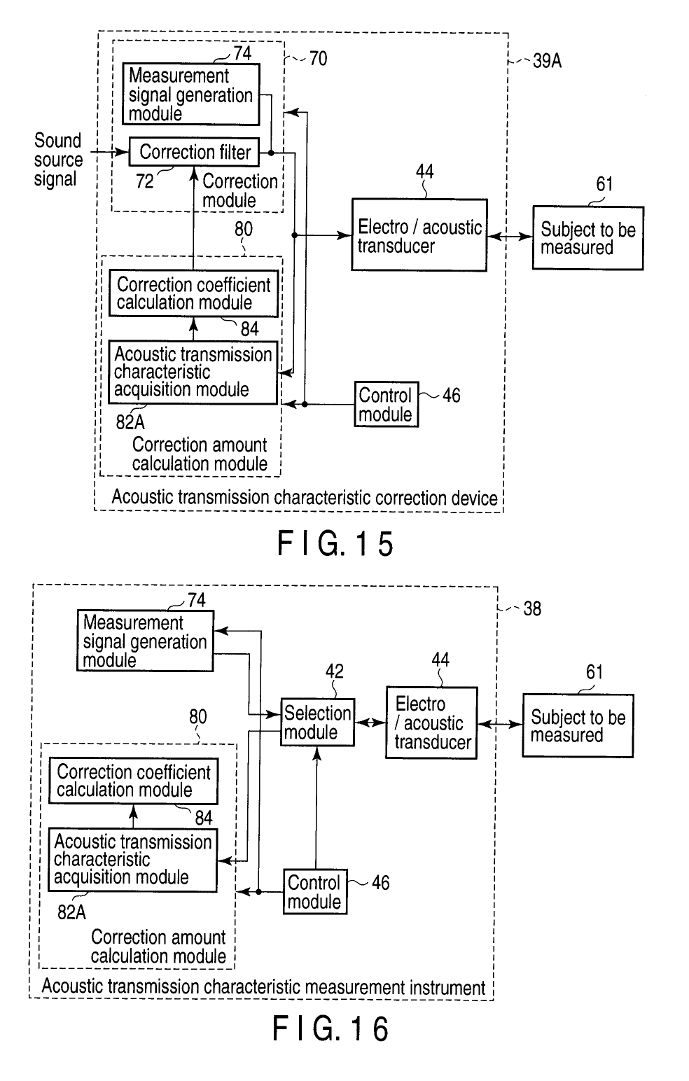

FIG. 15 shows a block view of an acoustic transmission characteristic correction apparatus 39A of a sixth embodiment. The correction apparatus 39A of the sixth embodiment is configured by removing the selection module 42 from the correction apparatus 39 of the fifth embodiment. Thereby, the output from the correction module 70 is supplied to the calculation module 80 as well as to the transducer 44. However, in the same way as that of the second embodiment, since the control module 46 controlling the operation timing recognizes whether the switching signal shown in FIG. 4C has a high level (a period in which the reference signal is output from the correction module 70), or has a low level (a reception period of the response signal from any portion of the subject 61 to be measured), the calculation module 80 may separate based on the signal from the control module 46, in chronological order, the reference signal for measurement output from the correction module 70 and the response signal that is the reflection signal from the subject 61, and obtain the acoustic transmission characteristic, correctly based on the response signal output from the transducer 44 not based on the reference signal output from the correction module 70 of the calculation module 80.

FIG. 16 shows a block diagram of an acoustic transmission characteristic measurement apparatus 38 of a seventh embodiment. The measurement apparatus 38 of the seventh embodiment is configured by removing the correction filter 72 from the correction apparatus 39 of the fifth embodiment.

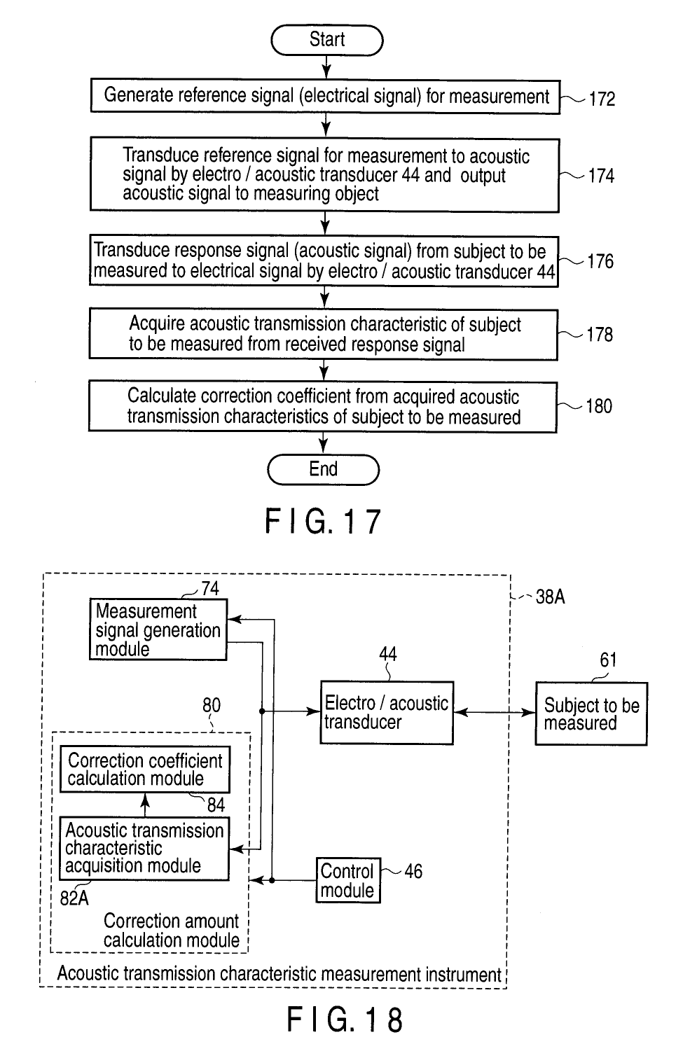

FIG. 17 shows a flowchart illustrating operations of the control module 46 of the measurement apparatus 38 of FIG. 16. The measurement signal generation module 74 generates the reference signal for measurement to be supplied to the selection module 42 (Block 172). At this moment, the selection module 42 has been brought into the first state as shown in FIG. 4C, the output (the reference signal for measurement as shown in FIG. 4A) from the generation module 74 is output into the subject 61 as the acoustic signal through the transducer 44 (Block 174). As shown in FIG. 4C, the selection module 42 switches into the second state after this, the response signal (FIG. 4B) that is the reflection signal of the acoustic signal to the reference signal for measurement reflected from any portion of the subject 61 to be measured is transduced into the electrical signal through the transducer 44, and the electrical signal is input to the correction amount calculation module 80 (Block 176).

In Block 178, the acquisition module 82A acquires the acoustic transmission characteristic in response to the received response signal. In Block 180, the calculation module 84 (i) transforms the acquired acoustic transmission characteristic from a time domain into a frequency domain, (ii) detects resonance peaks on a frequency axis, and (iii) calculates a coefficient of the correction filter so as to form dips of frequencies at which peaks are generated for canceling the detected peaks. The calculation of the coefficient may be performed by using a parametric equalizer and a graphic equalizer.

The calculated coefficient is provided for the separately disposed correction filter.

Thereby, a measurement apparatus which measures resonance characteristic and calculates the correction coefficient of the correction filter, and a correction apparatus to which the calculated correction coefficient is prepared to filter the sound source signal may be structured as separate units.

The measurement apparatus 38A may not include the correction coefficient calculation module 84. In this case, the flowchart of FIG. 17 performs the processes up to the acquisition of the resonance characteristic in Block 178, and the calculation of the correction coefficient in Block 180 is performed by the correction coefficient calculation module included in another apparatus.

FIG. 18 shows a block diagram of an acoustic transmission characteristic measurement apparatus 38A of an eighth embodiment. The measurement apparatus 38A is configured by removing the selection module 42 from the measurement apparatus 38 of the seventh embodiment. In the same way as that of the fourth embodiment, also in the eighth embodiment, the calculation module 80 may separate, in chronological order, the reference signal for measurement output from the measurement signal generation module 74 and the response signal that is the reflection signal from the subject 61 to be measured to the reference signal, correctly obtain the acoustic transmission characteristic.

As mentioned above, according to the embodiments of the invention, the correction apparatus can transfer the response acoustic signal from the subject to be measured to the reference signal for measurement into a response electrical signal by using the electro/acoustic transducer transducing the sound source signal into the acoustic signal, acquires the acoustic characteristic of the subject to be measured from the electrical signal, and calculates the correction coefficient of the correction filter of the sound source signal in response to the acquisition result. Then, the correction apparatus can precisely measure and correct the acoustic characteristic of the subject to be measured with a simple structure without making the transducer large and complex.

While certain embodiments of the inventions have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The various modules of the systems described herein can be implemented as software applications, hardware and/or software modules, or components on one or more computers, such as servers. While the various modules are illustrated separately, they may share some or all of the same underlying logic or code. The accompanying claims and their equivalents are intended to cover such forms or modifications as would fall within the scope and spirit of the inventions. For example, processing of averaging a plurality of response signals through a plurality of times of transmissions of the reference signal for measurement shown in FIGS. 5A-5C to acquire the acoustic characteristic of the subject to be measured may be applicable to all embodiments. The invention may be implemented as a computer-readable recording medium with a program for making a computer execute a prescribed means, for making the computer function as a prescribed means, and for making the computer achieve a prescribed function recorded thereon.

What is claimed is:

1. An acoustic characteristic correction apparatus comprising:
a correction module comprising a generation module configured to generate a measuring pulse for measuring an acoustic characteristic of a subject, and a correction filter configured to correct a sound source signal to be supplied to the subject;
a calculation module comprising an acquisition module configured to acquire an acoustic characteristic of the subject based on a response signal from the subject, and a calculation unit configured to calculate a correction coefficient of the correction filter based on the acoustic characteristic; and
a transducer module configured to transduce an output signal from the correction module into an acoustic signal to be transmitted to the subject, and an acoustic response signal from the subject into an electrical response signal to be supplied to the calculation module.

2. The acoustic characteristic correction apparatus of claim 1, wherein
the acquisition module is configured to separate the measuring pulse and a response signal from the subject, and acquire an acoustic characteristic of the subject based on the response signal, and further comprising:
a control module is configured to control the correction module or the calculation module when the acoustic characteristic is measured or the sound source signal is transmitted to the subject.

3. The apparatus of claim 2, wherein the transducer module is configured to comprise an earphone or a headphone.

4. The apparatus of claim 2, wherein the generation module is configured to generate a unit pulse or a time stretched pulse having a prescribed width at a plurality of times, and the correction module is configured to acquire the acoustic characteristic based on a plurality of electrical response signals output from the transducer module and calculate the correction coefficient based on the acquired acoustic characteristic.

5. The acoustic characteristic correction apparatus of claim 1, wherein
the acquisition module is configured to receive a response signal from the subject and acquire an acoustic characteristic of the subject based on the received response signal, and further comprising:
a selection module configured to supply the measuring pulse to the transducer module and supply the electrical response signal output from the transducer module to the calculation module when the acoustic characteristic is measured, and output a signal in which the sound source signal is corrected by the correction filter when the sound source signal is transmitted to the subject; and
a control module configured to control the selection module when the acoustic characteristic is measured or the sound source signal is transmitted to the subject.

6. The apparatus of claim 5, wherein the transducer module comprises an earphone or a headphone.

7. The apparatus of claim 5, wherein the generation module is configured to generate a unit pulse or a time stretched pulse having a prescribed width at a plurality of times, and the acquisition module is configured to acquire the acoustic characteristic based on a plurality of electrical response signals output from the transducer module and calculates the correction coefficient based on the acquired acoustic characteristic.

8. The apparatus of claim 5, wherein the selection module comprises a switch which is settable to a first state or a second state, connects the correction module and the transducer module in the first state, and connects an input of the calculation module and the transducer module in the second state.

9. An acoustic characteristic measurement apparatus comprising:
a generation module configured to generate a measuring pulse for measuring an acoustic characteristic of a subject;
an acquisition module configured acquire an acoustic characteristic of the subject based on a response signal from the subject; and
a transducer module configured to transduce the measurement pulse into an acoustic signal to be transmitted to the subject and transduce an acoustic response signal from the subject into an electrical response signal to be supplied to the acquisition module.

10. The acoustic characteristic measurement apparatus of claim 9, wherein
the acquisition module is configured to separate the measuring pulse and a response signal from the subject, and acquire an acoustic characteristic of the subject based on the response signal.

11. The apparatus of claim 10, wherein the transducer module comprises an earphone or a headphone.

12. The apparatus of claim 10, wherein the generation module is configured to generate a unit pulse or a time stretched pulse having a prescribed width at a plurality of times, and the acquisition module is configured to acquire the acoustic characteristic based on a plurality of electrical response signals output from the transducer module.

13. The acoustic characteristic measurement apparatus of claim 9, wherein
the acquisition module is configured to receive a response signal from the subject and acquire an acoustic characteristic of the subject based on the received response signal, and further comprising:
a selection module configured to supply the measurement pulse to the transducer module and then supply the electrical signal output from the transducer module to the acquisition module.

14. The apparatus of claim 13, wherein the transducer module comprises an earphone or a headphone.

15. The apparatus of claim 13, wherein the generation module is configured to output a unit pulse or a time stretched pulse having a prescribed width at a plurality of times, and the acquisition module is configured to acquire the acoustic characteristic based on a plurality of electrical response signals output from the transducer module.

16. The apparatus of claim 13, wherein the selection module comprises a switch which is settable to a first state or a second state, connects the correction module and the transducer module in the first state, and connects an input of the acquisition module and the transducer module in the second state.

17. An acoustic characteristic measurement method comprising:
generating a measurement acoustic signal;
transmitting the acoustic signal to a subject;
receiving an acoustic response signal from the subject;
acquiring an acoustic characteristic of the subject based on the received acoustic signal; and
calculating a correction coefficient of a correction filter based on the acquired acoustic characteristic.
